(19) United States
(12) Patent Application Publication
(10) Pub. No.: US 2023/0194520 A1
(43) Pub. Date: Jun. 22, 2023

(54) MULTIPLEX BIOSENSOR FOR RAPID POINT-OF-CARE DIAGNOSTICS

(71) Applicant: HEMEMICS BIOTECHNOLOGIES, INC., Gaithersburg, MD (US)

(72) Inventors: David HO, McLean, VA (US); John WARDEN, Chevy Chase, MD (US)

(21) Appl. No.: 17/925,548

(22) PCT Filed: May 14, 2021
(86) PCT No.: PCT/US2021/032584

§ 371 Date: Nov. 15, 2022

Related U.S. Application Data
(60) Provisional application No. 63/135,337, filed on Jan. 8, 2021.
(60) Provisional application No. 63/025,689, filed on May 15, 2020.

(51) Int. Cl. G01N 33/569; G01N 27/02; G01N 27/07; G01N 33/543

(52) U.S. Cl. CPC G01N 33/569; G01N 27/023; G01N 27/07; G01N 33/54326

(57) ABSTRACT

The present disclosure relates to carbon based biosensors and biosensor systems. The disclosure further relates to methods of rapidly detecting a target material in a biological sample using the biosensor and biosensor systems described herein to characterize a pathogen's antigen profile and/or a subject's immune response to pathogen exposure.

This application claims the priority benefit of U.S. Provisional Patent Application Ser. No. 63/025,689, filed on May 15, 2020, and 63/135,337, filed on Jan. 8, 2021, which is hereby incorporated by reference in its entirety.

### FIELD

The present disclosure is directed to biosensors, systems, and methods capable of rapid detection of a target material in a biological sample.

### BACKGROUND

The COVID-19 public health emergency highlighted the nation's need for next-generation diagnostics that can be easily deployed in both traditional care environments and the field. Delayed results, inaccurate reporting, and in some cases, inaccessibility to testing stymied the reopening of the economy and encouraged the spread of COVID-19.

Rapid, cost-effective, and real-time biomarker measurements are essential steps toward realizing the goal of quickly and effectively diagnosing emerging illnesses, like COVID-19 and other viruses. Currently, many biological assays rely on labeled detector molecules and optical-based detectors for diagnosis. The cost and time delay associated with these methods radically impacts patient outcomes, as testing, consultation and treatment are typically spread over several interactions. An innovative point-of-care biosensor device that can provide rapid, accurate disease detection is urgently needed.

The present invention is directed to overcoming these and other deficiencies in the art.

### SUMMARY

A first aspect of the present disclosure is directed to a biosensor that comprises a substrate comprising a planar surface and a spatially defined array of active areas on the planar surface of the substrate. Each active area on the planar surface comprises a carbon material and at least two spaced electrodes, wherein the carbon material is deposited on the planar surface of the substrate between the at least two electrodes. The biosensor further comprises a plurality of pathogen proteins or peptides thereof, wherein different pathogen proteins or peptides thereof are positioned at different active areas and immobilized on the deposited carbon material of said active areas. Finally, an electrical connection comprising a plurality of electrical contacts, where each electrical contact is configured to transmit an electrical signal between the at least two electrodes of a single active area and the electrical connection is also included on the biosensor.

Another aspect of the disclosure relates to biosensor system for characterizing a subject's immune response to pathogen exposure. This system comprises an electronic reader that comprises a circuit for delivering a signal and a processing device for reading the signal. The system further includes a biosensor as described herein that is operatively connected to the electronic reader via the electrical connection of the biosensor and configured to receive the signal delivered by the circuit. The electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.

Another aspect of the disclosure is directed to a method of characterizing a subject's immune response to pathogen exposure. This method involves collecting a biological sample from a subject and providing a biosensor system as described herein. The method further involves delivering an electrical signal to the biosensor via the circuit of the electronic reader and determining a base resistance between the two or more electrodes at each active site on the biosensor. The biological sample from the subject is applied to the biosensor and a change in the base resistance between the two or more electrodes at each active site on the biosensor is identified as result of applying the biological sample. The method further involves characterizing the subject's immune response to said pathogen exposure based on the identified change in base resistance.

Another aspect of the present disclosure is directed to a biosensor that comprises a substrate comprising a planar surface and a spatially defined array of active areas on the planar surface of the substrate. Each active area on the planar surface comprises a carbon material and at least two spaced electrodes, wherein the carbon material is deposited on the planar surface of the substrate between the at least two electrodes. The biosensor further comprises a collection of binding molecules, wherein different binding molecules of the collection bind different pathogen proteins and wherein different binding molecules are positioned at different active areas and immobilized on the deposited carbon material of said active areas. Finally, an electrical connection comprising a plurality of electrical contacts, where each electrical contact is configured to transmit an electrical signal between the at least two electrodes of a single active area and the electrical connection is also included on the biosensor.

Another aspect of the disclosure relates to biosensor system for characterizing a pathogen's antigen profile. This system comprises an electronic reader that comprises a circuit for delivering a signal and a processing device for reading the signal. The system further includes a biosensor as described herein that is operatively connected to the electronic reader via the electrical connection of the biosensor and configured to receive the signal delivered by the circuit. The electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.

Another aspect of the disclosure is directed to a method of characterizing a pathogen's antigen profile. This method involves collecting a pathogen containing sample and providing a biosensor system as described herein. The method further involves delivering an electrical signal to the biosensor via the circuit of the electronic reader and determining a base resistance between the two or more electrodes at each active site on the biosensor. The pathogen containing sample is applied to the biosensor and a change in the base resistance between the two or more electrodes at each active site on the biosensor is identified as result of applying the biological sample. The method further involves characterizing the pathogen's antigen profile based on the identified change in base resistance.

The biosensor described herein harnesses the superior electric charge capabilities of graphene to deliver a nearly instantaneous (˜60 seconds), highly sensitive point-of-care testing platform capable of detecting up to a dozen unique antibody/antigen pairs from a single drop of saliva. This allows for testing, diagnosis, and treatment in one interaction, resulting in more accurate, effective treatment plans and vastly improved patient outcomes. Because the biosensor is a Bluetooth capable device, data can be collected and analyzed in real time, from anywhere in the world.

### BRIEF DESCRIPTION OF THE DRAWINGS

FIG. 1 is a schematic cross-sectional view of an active area on an exemplary biosensor device as described herein.

FIG. 2 is a schematic cross-sectional view of an active area on an exemplary biosensor device as described herein.

FIG. 3 is a top-down view of a section of a biosensor device as described herein showing an array of active areas.

FIG. 4 is a top-down view of a section of a biosensor device as described herein showing multiple arrays of active areas.

FIG. 5 is an electrical circuit diagram illustrating the electrical circuitry associated with an array of active areas on a biosensor device as described herein.

FIG. 6 is a schematic view of an exemplary electronic reader for use in combination with the biosensor described herein.

FIG. 7 is a block diagram of an exemplary circuit that may be used in combination with the electronic reader of FIG. 6.

FIG. 8 is a flowchart of an exemplary process for detecting a target moiety using a biosensor and electronic reader as described herein.

FIG. 9 is an image of PET substrate coated with a single layer of graphene spotted with silver paint to establish electrical connection.

FIG. 10 is a graph of circuit resistance over the 7-step adhesion process. During this process protein or antibody was immobilized on the surface of the graphene sensor.

FIGS. 11A-11C show the method of detecting target antigen in a sample using a biosensor containing an electromagnetic substrate as described herein. As shown in the cross-sectional view of the sensor provided in FIG. 11A, the electromagnet is positioned beneath the substrate of the biosensor, and the antibody (or other biological detecting agent) is immobilized to the active areas on the surface of the substrate. A drop of high viscosity fluid containing antigen complexed to magnetic beads is applied to active areas on the surface. In the depiction of FIG. 11A, the electromagnet is turned off. Absorption of magnetic bead-antigen complex onto the surface of active areas on the biosensor when the electromagnet is turned on is depicted in FIG. 11B. When the electromagnet is turned off, unbound magnetic bead-antigen complex is released from the circuit, whereas antigen-bead complex that is specifically bound to the immobilized antibody (or other biological detecting agent) will remain bound to the circuit and be detected (FIG. 11C).

FIG. 12 is a graph showing the change in dirac point voltage across two circuits containing electromagnets beneath the surface. The far left box shows the dirac point voltage at baseline. After 594 seconds, 2.5 nm-ferrous oxide magnetic beads conjugated to BSA were added to the circuit and a corresponding increase in signal was observed between 600-800 seconds (middle box). The transition stabilized at about 800 seconds, and a second addition of 2 nm-ferrous oxide magnetic bead conjugated to BSA was made at 1188 seconds. This addition let to another increase in voltage by about 200 mV over the course of 200 seconds (far right box).

### DETAILED DESCRIPTION

The present disclosure is directed to biosensors, systems, and methods capable of rapid detection of a target material in a biological sample.

In one aspect, the biosensor disclosed herein comprises a substrate that comprises a planar surface and a spatially defined array of active areas on the planar surface of the substrate. Each active area on the planar surface comprises a carbon material; and at least two spaced electrodes, wherein the carbon material is deposited on the planar surface of the substrate between the at least two electrodes. The biosensor further comprises a plurality of pathogen proteins or peptides thereof, wherein different pathogen proteins or peptides thereof are positioned at different active areas and immobilized on the deposited carbon material of said active areas; and an electrical connection comprising a plurality of electrical contacts, each electrical contact configured to transmit an electrical signal between the at least two electrodes of a single active area and the electrical connection.

In another aspect, the biosensor disclosed herein comprises a substrate that comprises a planar surface and a spatially defined array of active areas on the planar surface of the substrate. Each active area on the planar surface comprises a carbon material; and at least two spaced electrodes, wherein the carbon material is deposited on the planar surface of the substrate between the at least two electrodes. The biosensor further comprises a collection of binding molecules, wherein different binding molecules of the collection bind different pathogen proteins and wherein different binding molecules of the collection are positioned at different active areas and immobilized on the deposited carbon material of said active areas; and an electrical connection comprising a plurality of electrical contacts, each electrical contact configured to transmit an electrical signal between the at least two electrodes of a single active area and the electrical connection.

The basic structure of the biosensor disclosed herein is described in International Patent Application Publication No. WO2020072966 to Hememics Biotechnologies, Inc., which is hereby incorporated by reference in its entirety.

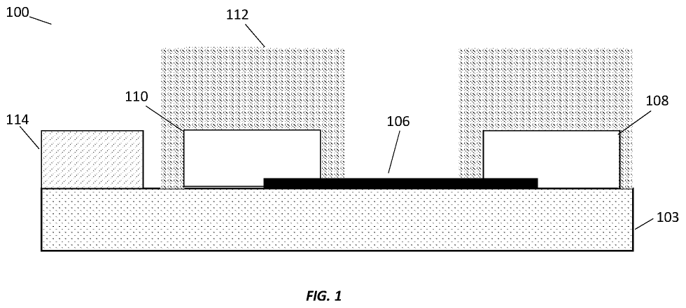

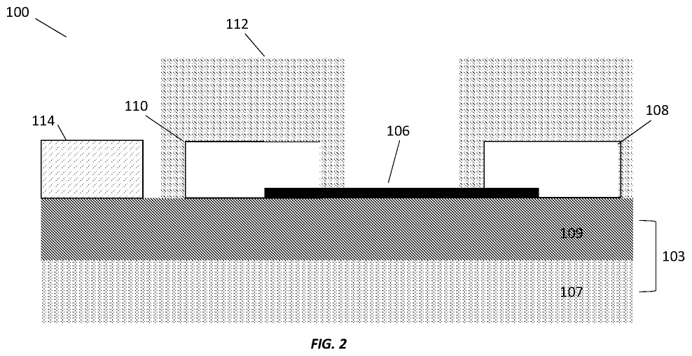

The schematic illustrations of FIGS. 1 and 2 provide cross-sectional views of an active area 100 on a biosensor as described herein. In reference to FIGS. 1 and 2, the biosensor comprises a substrate 103 having a planar surface. As shown in the embodiment of FIG. 1, the substrate can comprise a single-layer, or as shown in FIG. 2 the substrate can comprise two or more layers.

In some embodiments, the biosensor comprises a single-layer substrate. In accordance with this embodiment, the single-layer substrate is a polymeric material. Suitable polymeric materials include, without limitation, poly(methyl methacrylate) (PMMA), polycarbonates (PC), epoxy-based resins, copolymers, polysulfones, elastomers, cyclic olefin copolymer (COC), nylon, polypropylene, a polyester film (e.g., Melinex® 514P (Dupont)), polyethylene terephthalate and polymeric organosilicons. In any embodiment, the polymeric substrate is modified with an anti-static agent to exhibit suitable anti-static properties to dissipate electrical charge. The anti-static agent can be mixed directly with the polymer material or applied to the surface of the polymer material to impart anti-static quality to the material. Anti-static agents that can be added to polymers to minimize static electricity are known in the art and include, without limitation, fatty acid esters, long chain aliphatic amines and amides, ethoxylated amines, quaternary ammonium compounds (e.g., behentrimonium chloride or cocamidopropyl betaine), esters of phosphoric acid, polyethylene glycol esters, alkylsulfonates, and alkylphosphates. A suitable anti-static quality for a substrate of the biosensor as described herein is determined by the surface resistivity of the material, which is measured in ohms/square. Suitable anti-static polymeric substrate materials of the biosensor have a surface resistivity of between 10³-10¹⁰ ohms/square. In some embodiments, the anti-static polymeric substrate material of the biosensor has a surface resistivity of between 10³-10⁵ ohms/square.

Alternatively, as shown in FIG. 2, the substrate of the biosensor may comprise two or more layers. For example, the substrate 103 can comprise a first layer 107 and a second layer 109. In accordance with this embodiment, suitable substrate materials comprise glass, silica, fused silica, quartz, and composite materials. In one embodiment, the biosensor comprises a first conductive layer 107, e.g., a layer of highly doped silicon and a second, insulating dielectric layer 109.

In reference to FIGS. 1 and 2, each active area 100 on a biosensor functions as a field-effect transistor (FET) sensor unit with a liquid gate. Each active area comprises a conductive carbon material 106 (e.g., graphene) deposited on the planar surface of the substrate 103 between at least two spaced electrodes 108, 110. Suitable conductive carbon materials of the active areas include, without limitation, graphene, carbon nanotubes, fullerene or a combination thereof. The area between the electrodes can alternatively comprise other conductive materials known in the art, including, without limitation, silicene, molybdenum disulfide, black phosphorous, and/or metal dichalcogenides.

The spaced electrodes comprise a first electrode 108, that serves as a source electrode, and the second electrode 110, that serves as a drain electrode. The electrodes each comprise a conductive metal, such as, for example and without limitation gold (Au), copper (Cu) silver (Ag), cobalt (Co), platinum (Pt), and combinations thereof. As show in FIGS. 1 and 2, the electrodes can be encapsulated in an insulating material 112. Each active area of a biosensor further comprises a gate conductor 114, which controls the liquid gate, i.e., the electrical field in the ionic fluid sample that is applied to the graphene surface 106 during use of the sensor.

A collection of biological detecting agents, for example, a collection of binding molecules or a plurality of pathogen proteins or peptides as described herein, are immobilized to the surface of the carbon material 106, preferably in the presence of a preservative solution as disclosed herein. Suitable biological detecting agents, preservative agents, and methods of immobilizing the biological detecting agents to the surface of the carbon material are described herein.

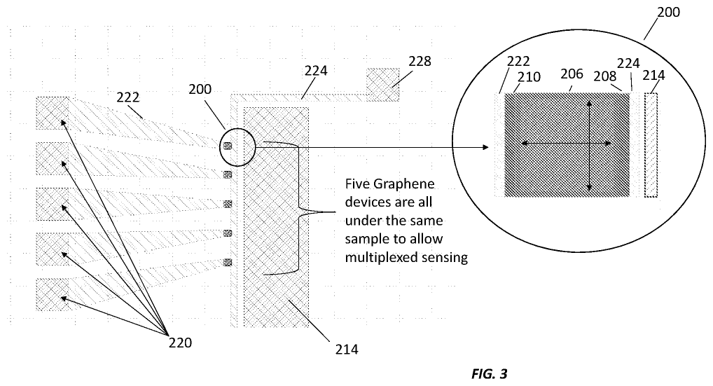

A top-down view of a section of the biosensor device is provided in FIG. 3. This illustration shows a series of five active areas 200, each active area 200 comprising the conductive carbon material 206 deposited on the substrate. The conductive carbon material 206 forms a channel between the source 208 and drain 210 electrodes of the active area, and is in close proximity to the gate conductor 214. Exemplary dimensions, i.e., length and width, of the carbon material channel of the active area may range from about 10 microns to about 250 microns in width and from about 10 microns to about 250 microns in length. For example, the channel length may range from about 10 microns to about 250 microns, from about 25 microns to 200 microns, from about 50 microns to about 150 microns, from about 75 microns to about 100 microns. In some embodiments, the channel length is about 75 microns, about 80 microns, about 90 microns, or about 100 microns. In some embodiments, the channel length is about 90 microns. Similarly, the width of the channel from side to side may range from about 10 microns to about 250 microns, from about 25 microns to 200 microns, from about 50 microns to about 150 microns, from about 75 microns to about 100 microns. In some embodiments, the channel width is about 75 microns, about 80 microns, about 90 microns, or about 100 microns. In some embodiments, the channel width is about 90 microns. As shown in this embodiment, the gate conductor 214 controls the liquid gate, i.e., the electrical field in the ionic fluid sample applied to each graphene surfaces 206 on the device, for all five active areas 200, although other numbers of active areas may be employed.

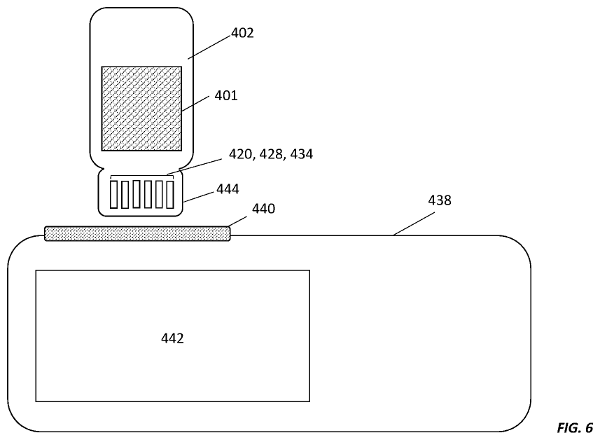

The biosensor further comprises an electrical connection for operatively connecting the biosensor to an electronic reader. The electrical connection comprises a plurality of electrical contacts where each contact is capable of transmitting an electrical signal between the electrodes of each active area and the electrical connection. In reference to FIG. 3, the electrical contacts include, the shared bonding pads 220 and shared source pad 228. For example, the electrical connection provides current (i.e., electrical signal) from the reader, as described below, to each of a plurality of active areas on the biosensor via the shared source pad 228 and shared source line 224 (i.e., conductive wire). After passing through the source 208 and drain 210 electrodes of an active area, the current is transmitted to the electrical connection via the individual drain lines 222 and drain bonding pads 220. The drain bonding pads 220 of the electrical connection form a circuit with components of the reader device (illustrated in FIG. 6), as described below.

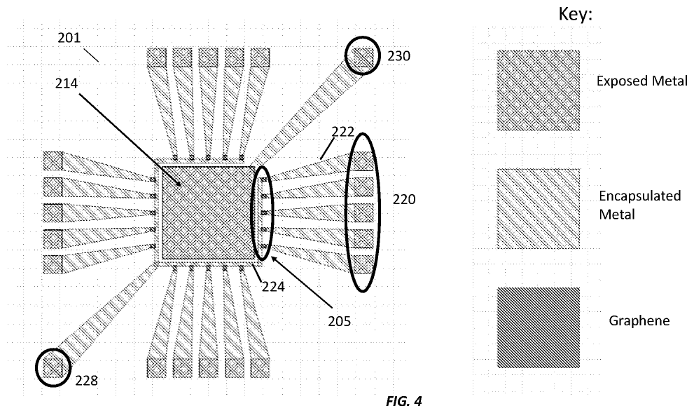

A top-view of a sensing unit 201 of the biosensor is provided in FIG. 4. The sensing unit comprises all of the active areas 200 on a biosensor. In this illustration, the sensing unit 201 of the biosensor comprises four arrays 205 of active areas 200, each array 205 comprising five active areas 200 (as shown in FIG. 3), although other numbers of active areas may be employed. The arrays of active areas are arranged around the periphery of the gate conductor 214. In some embodiments, the gate conductor is at least one order of magnitude greater in size than the dimensions of the carbon material of the active area on the sensor. In some embodiments, the gate conductor is at least two orders of magnitude greater in size than the dimensions of the carbon material of the active area. In some embodiments, the gate conductor is at least three orders of magnitude greater in size than the dimensions of the carbon material of the active area. The larger dimensions of the gate conductor relative to the carbon material of the active area increases the sensitivity of the sensor to detecting voltage changes. Current is fed to each of the twenty active areas 200 via the electrical connection through the shared source pad 228 and shared source line 224. Current is provided to the gate conductor 214 via the electrical connection through the conductor gate bonding pad 230. Any change in current flow through the active area, e.g., an increase in resistance resulting from the presence and binding of a target moiety in a sample applied to the sensor to the immobilized biological detecting agent on the surface of the graphene, is transmitted to the electrical connection of the sensor by the individual drain lines 222 and drain bonding pads 220.

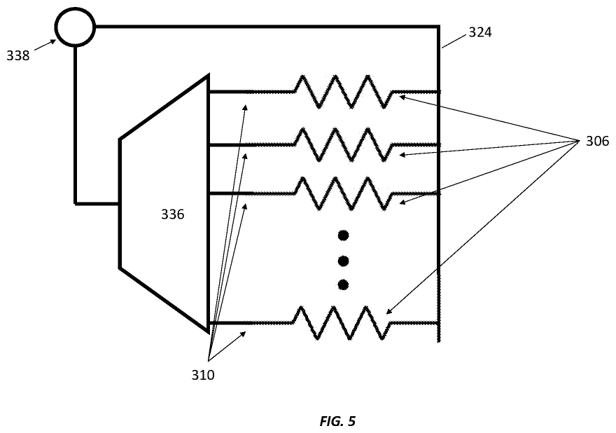

FIG. 5 shows an electrical circuit diagram illustrating aspects of FIG. 4. In this diagram, the graphene active areas 306 are modeled as resistors, receiving current from the shared source 324. In this illustration, the individual drain lines and drain pads connect to a multiplexer 336. The multiplexer 336 acts as a selector, selectively retrieving signal and transmitting the signal to the electrical connection for further transmission to a detector 338 in the reader.

As shown in FIG. 4, the sensing unit of a biosensor as disclosed herein comprises a plurality of active areas on the planar surface of the substrate to facilitate multiplex detection of different target moieties. As will be understood by one of skill in the art, a biosensor as described herein may comprise at least 10, at least 20, at least 30, at least 40, at least 50, at least 60, at least 70, at least 80, at least 90, at least 100, at least 125, at least 150, at least 175, at least 200, at least 225, at least 250, at least 275, at least 300, at least 325, at least 350, at least 375, at least 400, at least 425, at least 450, at least 475, at least 500 or more active areas.

Deposited on the carbon or other conductive carbon material of each active unit is a collection of biological detecting agents. In one embodiment, the active units across the biosensor device each contain a collection of different biological detecting agents, where the detecting agents are pathogen proteins or peptides thereof. In accordance with this embodiment, each of the different pathogen proteins or peptides thereof are positioned at different active areas and immobilized on the deposited carbon material of said active areas across the biosensor surface. In some embodiments, the plurality of pathogen proteins or peptides are derived from one or more infectious agents selected from a virus, a bacterium, or a combination thereof. Suitable pathogen proteins or peptide for immobilizing on the graphene surface are generally between 5 and 100 amino acid residues in length, 5 and 75 amino acid residues in length, 5 and 50 amino acid residues in length, 10 and 50 amino acid residues in length, 15 and 50 amino acid residues in length, 20 and 50 amino acid residues in length, 25 and 50 amino acid residues in length, 30 and 50 amino acid residues in length, 35 and 50 amino acid residues in length, 40 and 50 amino acid residues in length, 45 and 50 amino acid residues in length, 45 and 75 amino acid residues in length, or 45 and 100 amino acid residues in length.

In some embodiments, the plurality of pathogen proteins or peptides are derived from one or more viruses, including, but not limited to SARS-CoV-2, Influenza A, Influenza B, Human papilloma virus, Venezuelan equine encephalitis virus, Vaccinia virus, Ebola virus, Lassa fever virus, Rift Valley fever virus and combinations thereof. The pathogen proteins or peptides can also be derived from parainfluenza, paramyxovirus, adenovirus, parvovirus, enterovirus, variola virus, rotavirus, hemorrhagic fever viruses (viruses in the families of Arenaviridae, Bunyaviridae, Filoviridae, Falviviridae, and Togaviridae) hepatitis virus, parechovirus, human T-lymphotrophic virus, and Epstein-Barr virus (herpes virus).

In one embodiment, the plurality of pathogen proteins or peptides are derived from coronavirus. These include both human coronaviridae virus (e.g., SARS-CoV-2, SARS-CoV, MERs-CoV, HCoV-NL63, HCoV-229E, HCoV-OC43, and HCoV-HKU1) and animal coronaviridae viruses (e.g., Feline CoV [serotypes I and II], porcine epidemic diarrhea CoV (PEDV), porcine PRCV, porcine TGEV, Dog CCOC, Rabbit RaCoV, etc.).

In some embodiments, the plurality of pathogen proteins or peptides are derived from different viruses so as to allow for the multiplex detection of different corresponding antibodies in a biological sample being tested. In some embodiments, the plurality of pathogen proteins or peptides are derived from the same virus, e.g., SARS-CoV-2, to comprehensively characterize a subject's immune (i.e., antibody) response to infection by the virus. In some embodiments, the plurality of pathogen proteins or peptides are derived from SARS-CoV-2. In some embodiments, the plurality of pathogen proteins or peptides are derived from SARS-CoV-2 and Influenza A.

In some embodiments, the plurality of pathogen proteins or peptides are derived from one or more bacteria, including, but not limited to Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, Enterococcus faecium, Staphylococcus aureus, Klebsiella pneumonia, Acinetobacter baumannii, Enterobacter species and combinations thereof.

In one embodiment, the collection of biological detecting agents immobilized on the deposited material of said active area comprises a collection of binding molecules. Suitable binding molecules for immobilization on the active areas of the biosensor encompass any biological material that serves as a binding partner or pair to a detectable target material present or potentially present in a biological sample. In some embodiments, the binding molecules of the collection are antibody-based molecules. An antibody-based molecule as used herein includes, without limitation full antibodies, epitope binding fragments of whole antibodies, and antibody derivatives.

Full antibodies include intact immunoglobulins comprising two heavy chains and two light chains, each of these chains comprising a variable region (i.e., V_{H} and V_{L}) and constant region (i.e., C_{H} and C_{L}). Epitope binding fragments of antibodies (including Fab and (Fab)₂ fragments) that exhibit epitope-binding that are suitable for immobilization on the active area of the biosensor include without limitation (i) Fab′ or Fab fragments, which are monovalent fragments containing the V_{L}, V_{H}, C_{L} and C_{H}1 domains; (ii) F(ab′)₂ fragments, which are bivalent fragments comprising two Fab fragments linked by a disulfide bridge at the hinge region; (iii) Fd fragments consisting essentially of the V_{H} and C_{H}1 domains; (iv) Fv fragments consisting essentially of a V_{L} and V_{H} domain, (v) dAb fragments, which consist essentially of a V_{H} or V_{L} domain and also called domain antibodies, and (vii) isolated complementarity determining regions (CDR). An epitope-binding fragment may contain 1, 2, 3, 4, 5 or all 6 of the CDR domains of such antibody. Antibody derivatives suitable for immobilization on the active areas of the biosensor include those molecules that contain at least one epitope-binding domain of an antibody, and are typically formed using recombinant techniques. One exemplary antibody derivative includes a single chain Fv (scFv). A scFv is formed from the two domains of the Fv fragment, the V_{L} region and the V_{H} region.

In some embodiments, the binding molecules of the collection are antibody mimetics. Exemplary antibody mimetics for immobilization on the biosensor active areas are readily known in the art and include, without limitation, affibodies, affilins, affimers, monobodies, and DARPINs.

Other binding materials suitable for immobilization on the carbon material of the active areas of the biosensors described herein includes, without limitation, carbohydrates, lipids, nucleic acids (DNA, RNA), recombinant proteins, hybrid molecules such as protein conjugated to DNA or RNA, DNA conjugated to carbohydrates, etc. Biological binding materials also encompass, for example, whole cells or cell fragments of mammalian cells, prokaryotic cells, parasites, viruses, nucleated or enucleated cells.

The collection of binding molecules immobilized on the carbon surface of the active areas of the biosensor bind one or more pathogenic proteins, including pathogenic proteins from infections agents such as viruses, bacteria, toxins, and combinations thereof. Detection of the pathogenic proteins in a sample via binding to the binding molecules is indicative of the presence of the pathogen in the sample. In some embodiments, the binding molecules of the collection on the biosensor bind to one or more pathogenic proteins from a single infectious agent. In some embodiments, the collection of binding molecules on the biosensor bind to pathogenic proteins from different infectious agents to enable multiplex detection of various pathogens in a single sample (e.g., multiplex detection of viruses, bacteria, and/or toxins).

In some embodiments, the binding molecules of the collection bind one or more pathogenic proteins of a virus. Exemplary viruses include, without limitation, SARS-CoV-2, influenza A, influenza B, human papilloma virus, Venezuelan equine encephalitis virus, Vaccinia virus, Ebola virus, Lassa fever virus, Rift Valley fever virus and combinations thereof. In some embodiments, binding molecules of the collection bind one or more pathogenic proteins of SARS-CoV-2. In some embodiments, binding molecules of the collection bind one or more pathogenic proteins of SARS-CoV-2 and Influenza A.

In some embodiments, binding molecules of the collection bind one or more pathogenic proteins of bacteria. Exemplary bacteria include, without limitation, Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.

In some embodiments, binding molecules of the collection bind one or more toxins to facilitate detection of the presence of a toxin in a biological sample. Exemplary toxins that can be detected using suitable antibodies include, without limitation, Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.

In some embodiments, the collection of biological detecting agents immobilized on the deposited material of said active area (e.g., the carbon material) comprises a collection of binding molecules together with a plurality of pathogenic proteins, where different binding molecules and pathogenic proteins are spatially arranged at different active areas on the biosensor surface. The combination of binding molecules (suitable for detecting the presence of pathogenic proteins in a sample) and pathogenic proteins or peptides (suitable for detecting the presence of antibodies in a sample) enables a comprehensive characterization of a biological sample. For example, when the sample is a biological sample from a subject (e.g., a human mucosal, blood, or plasma sample), detecting the presence of pathogenic proteins in the sample indicates the presence of an active infection while detecting the presence of antibodies in the sample indicates previous infection and/or provides information on the immune response mounted against that infection.

In some embodiments, the active areas containing the biological detecting agents, i.e., the pathogenic proteins or peptide thereof or binding molecules, are contacted with a preservative solution to preserve the integrity and stability of the biological detecting agents immobilized thereto. In some embodiments, the preservative solution comprises at least one large MW sugar (>40,000 Da) and at least another smaller MW sugar (<40,000 Da). Once added to the active areas containing the detecting agents, the detecting agents are dried to a final moisture content of from about 5% to about 95%. At least one large MW sugar and the at least another small MW sugar can be present in a single preservative solution or may be separate solutions.

In some embodiments, the preservative solution included in the deposited material comprises at least one membrane penetrable sugar, at least one membrane impenetrable sugar, at least one anti-microbial agent, at least one anti-oxidant, optionally a salt, adenosine, and, optionally, albumin. In some embodiments, the preservative solution comprises at least one membrane penetrable sugar (e.g., trehalose and glucose), at least one membrane impenetrable sugar (e.g., dextran, such as dextran-70), at least one anti-microbial agent (e.g., sulfanilamide), at least one anti-oxidant (e.g., mannitol and vitamin E), optionally adenosine, and, optionally, albumin. In some embodiments, the preservative solution comprises at least one membrane penetrable sugar (e.g., trehalose and glucose), at least one membrane impenetrable sugar (e.g., dextran, such as dextran-70), at least one anti-microbial agent (e.g., sulfanilamide), at least one anti-oxidant (e.g., mannitol and vitamin E), adenosine, albumin, a salt (e.g., chloride salts such as KCl, NaCl, CaCl2, and covalent chlorides of metals or nonmetals such as titanium(IV) chloride or carbon tetrachloride), a buffer (e.g., K2HPO4), and a chelating agent (e.g., EDTA). Suitable preserving liquids and methods are described in U.S. Pat. Nos. 8,628,960, 9,642,353, and 9,943,075, each of which is incorporated herein in its entirety, or available from HeMemics Biotechnologies, Inc. (Hem Sol™).

In accordance with present disclosure, each of the plurality of biological detecting agents, i.e., pathogen proteins or peptides and/or binding molecules are immobilized on the deposited carbon material. In some embodiments, the immobilization is via a covalent bonding interaction. In some embodiments, the binding molecule and/or proteins or peptides are attached via a hydrophobic linker, wherein the hydrophobic linker is coupled to the detecting agent's amino or carboxy terminus. In some embodiments, the hydrophobic linker is a peptide linker comprising two or more linker amino acids and one or more aromatic amino acid residues. In some embodiments, the two or more linker amino acid residues are selected from glycine, alanine, serine, and combinations thereof. In some embodiments, the hydrophobic linker comprises a polycyclic aromatic hydrocarbon. A suitable polycyclic aromatic hydrocarbon linker comprises, without limitation, pyrene.

Other methods of immobilizing the biological detecting agents to the carbon material of the active areas are known in the art and suitable for use in accordance with the biosensor described herein. These include, for example, and without limitation, attachment via 1-ethyl-3-(3-dimethylamino propyl carbodiimide hydrochloride (EDC) and N-hydroxysuccinimide (NETS) (EDC/NHS) chemical reactions, attachment via electrostatic bonding, or via 1-pyrenebutanoic acid succinimidyl ester (PASE) linker (see e.g., Pena-Bahamonde et al., “Recent Advances in Graphene-based Biosensor Technology with Applications in Life Science,” J. Nanobiotechnology 16:75 (2018), which is hereby incorporated by reference in its entirety).

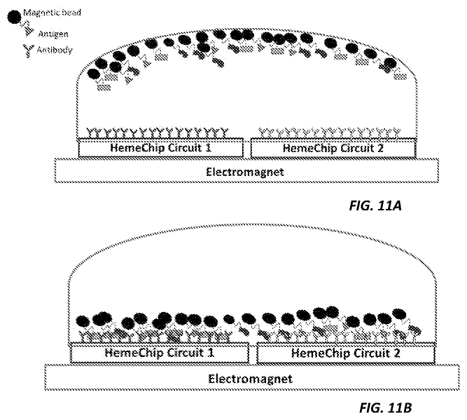

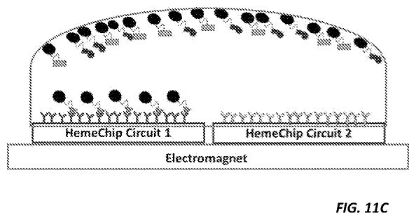

In one aspect, the biosensor of the present disclosure further comprises an electromagnet that is positioned beneath the substrate of the biosensor. The biosensor further comprises a means for turning the electromagnet on and off. A schematic of a biosensor comprising an electromagnet and the sequential steps of sample antigen or antibody detection using the electromagnet is provided in FIGS. 11A-11C.

As described herein, the electromagnet feature of the biosensor used in combination with a sample, where the antigens or antibodies of the sample have been conjugated to magnetic beads, allows user control over the rate of sample antigen/antibody diffusion to the surface of the biosensor, ensuring quick absorption of the antigen/antibody to the active areas of the biosensor surface. This ensures antigens/antibodies in the sample are brought in close proximity to the active areas containing the detecting agents on the surface of the sensor to facilitate binding between the detecting agent and target material (i.e., antigens/antibodies of the sample) if the target material is present in the sample. This reduces false negative results that may arise if diffusion alone is relied on. This is especially important feature to employ when testing samples where the target material may be present in very low concentrations.

Another aspect of the present disclosure is directed to a biosensor system. In one embodiment, the biosensor system is useful for characterizing a subject's immune response to pathogen exposure. In another embodiment, the biosensor system is useful for characterizing a pathogen's antigen profile. In either embodiment, the biosensor system comprises an electronic reader, where the electronic reader comprises a circuit for delivering a signal, and a processing device for reading the signal. The biosensor system further includes a biosensor as described herein that is operatively connected to the electronic reader and configured to receive the signal delivered by the circuit. The electronic reader is configured to deliver the signal to the biosensor and obtain an output impedance value before and after a sample has been applied to the array of active areas on the biosensor. The processing device is configured to compare the output impedance values to determine whether a binding event has occurred at one or more of the active areas to characterize the subject's immune response to pathogen exposure when the active areas contain pathogenic proteins/peptides, or to determine the presence of a pathogen in a sample and characterize its antigen profile when the active areas contain binding molecules (e.g., antibodies or antibody-based molecules). In some embodiments, the system is capable of characterizing both the subject's immune response and determine the presence and antigenic profile of a pathogen in a sample when the active areas across the biosensor contain.

In some embodiments, the biosensor of the biosensor system comprises an electromagnet positioned beneath the substrate of the biosensor as described infra.

FIG. 6 is a schematic view of a biosensor system including an electronic reader 438 for receiving the biosensor 402. The electronic reader 438 may include a slot 440 for receiving the electrical connection 444 of the biosensor 402. Insertion of the biosensor 402 completes a circuit within the electronic reader 438 via the electrical connection 444 comprising a plurality of electrical contacts, i.e., the drain bonding pads 420, the shared source bonding pad 428, and the gate bonding pad 434. The electronic reader 438 may further include a user interface 442 for outputting information to a user. In some embodiments, the electronic reader 438 may provide signals to a user interface not present on the actual reader 438, e.g., via Bluetooth (or other communication means) to a monitor or other display.

In some embodiments, the biosensor systems as described herein further comprises a communication interface coupled to the electronic reader for transmitting data from the electronic reader, and a data management computing device configured to receive data from the electronic reader via the communication interface. In accordance with this embodiment, the data management computing device comprises a memory coupled to a processor which is configured to execute programmed instructions comprising and stored in the memory to geographically map immune response data to pathogen exposure and or pathogen antigenic profile data (i.e., the presence or evolution of various pathogen strains), based on data received from electronic reader.

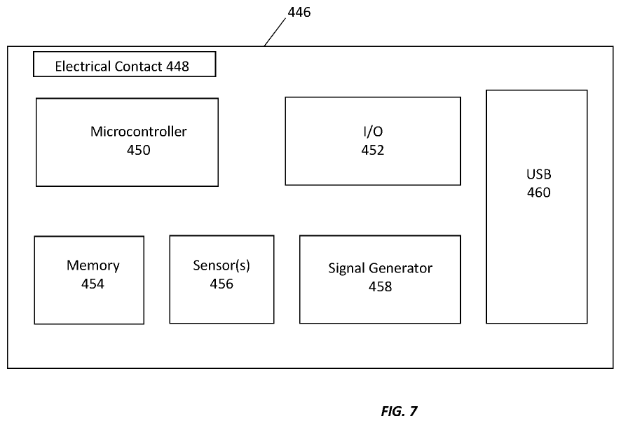

FIG. 7 is an exemplary embodiment of this aspect of the disclosure showing a block diagram of a circuit 446, such as a circuit board with computing components for providing a signal to the biosensor 402 and receiving a return signal to test a sample placed on the biosensor 402. In an exemplary embodiment, the circuit 446 includes a contact 448 which may be an electrical contact for interacting with the electrical connection 444 containing the electrical contacts 420, 428, 434 of the biosensor 402 to complete a circuit that includes the electronic reader 438 and the biosensor 402.

The circuit 446 may also include computing components including, but not limited to, a microcontroller 450, one or more I/O devices 452, a memory or other storage component 454, one or more sensors 456, a signal generator 458, and a USB or other communication hub 460. The computing components are exemplary and may be replaced with other components to execute disclosed embodiments for testing a sample via the biosensor 402.

The microcontroller or processor 450 may be a processing device configured to monitor and control components of the circuit 446, such as to perform setup, testing, and output processes via the electronic reader 438. The processor 450 may execute programmed instructions stored in the memory 454 for any number of functions described and illustrated herein. The processor 450 may include one or more central processing units (CPUs) or general purpose processors with one or more processing cores, for example, although other types of processor(s) can also be used.

The memory 454 of the electronic reader 438 stores these programmed instructions for aspect(s) of the present technology as described and illustrated herein, although some or all of the programmed instructions could be stored elsewhere. A variety of different types of memory storage devices, such as random access memory (RAM), read only memory (ROM), hard disk, solid state drives (SSD), flash memory, or other computer readable medium which is read from and written to by a magnetic, optical, or other reading and writing system that is coupled to the processor(s) 450, can be used for the memory 454.

The I/O device(s) 452 may include the communication interface 442, for example, to obtain input and provide output to and from a user. The communication interface 442 of the electronic reader 438 operatively couples and communicates between at least the electronic reader 438 and an external computing device, in some embodiments, which are coupled together at least in part by one or more communication network(s) or a public cloud network. By way of example only, the communication network(s) can include local area network(s) (LAN(s)) or wide area network(s) (WAN(s)) and the public cloud network can include a WAN (e.g., the Internet). The communication network(s) and/or the public cloud network can use TCP/IP over Ethernet and industry-standard protocols, although other types or numbers of protocols or communication networks can be used. The communication network(s) and/or public cloud network in this example can employ any suitable interface mechanisms and network communication technologies including, for example, Ethernet-based Packet Data Networks (PDNs) and the like.

The sensors 456 may include a voltage divider, resistance sensor, impedance sensor, or other device configured to determine a value associated with an electrical property at one or more locations on the biosensor 402. The signal generator 458 may be configured to generate an AC electrical signal for delivery to the biosensor 402. The USB port 460 may be a connection element for receiving and providing data exterior to the electronic reader.

The biosensor and biosensor systems described herein can be utilized to analyze a number of different biological samples to detect the presence of pathogen proteins and/or a subject's immune response to infection with a pathogenic organism or infectious agent. Accordingly, another aspect of the present disclosure is directed to a method of characterizing a subject's immune response to pathogen exposure. This method involves collecting a biological sample from a subject. A suitable sample is any biological fluid from the subject, including, without limitation, whole blood, blood serum, blood plasma, ascites fluid, cyst fluid, pleural fluid, peritoneal fluid, cerebrospinal fluid, tears, urine, saliva, sputum, lymph fluid, synovial fluid, amniotic fluid, follicular fluid, fluid of the respiratory, intestinal, and genitourinary trances.

The method further involves providing the biosensor system as described herein. Suitable biosensors include those containing a plurality of pathogen proteins and/or peptides immobilized at the active areas of the sensor. The method further involves delivering an electrical signal to the biosensor via the circuit of the electronic reader and determining a base resistance between the two or more electrodes at each active site on the biosensor. The method further involves applying the biological sample from the subject to the biosensor and identifying a change in the base resistance between the two or more electrodes at each active site on the biosensor resulting from said applying. The change in base resistance is indicative of an antibody from the sample binding to the immobilized pathogen proteins or peptides. The subject's immune response to pathogen exposure can be characterized based on the identified change in base resistance between the electrodes at the various active sites on the biosensor. Alternatively, the change in resistance is indicative of the presence of a pathogen in a sample, based on pathogen protein binding to the immobilized binding agents present in the active areas of the sensor. The pathogen's antigenic profile can be characterized based on the identified change in base resistance between the electrodes at the various active sites on the biosensor.

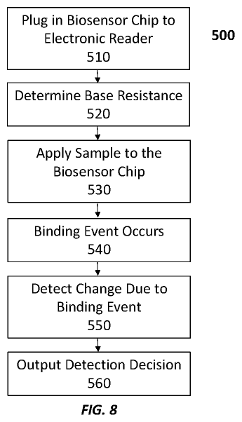

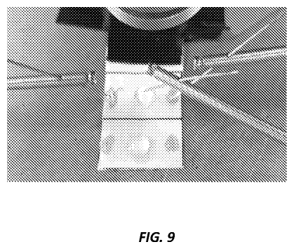

FIG. 8 provides an exemplary process 500 for detecting a target moiety using a biosensor 402 and electronic reader 438. The biosensor 402 is manufactured to include a collection of difference biological detecting agents as described supra.

An important feature of the biosensor device described herein relates to the arrangement of biological detecting agents across the sensing unit of the biosensor. In some embodiments, one or more active areas on the surface of the biosensor contains a collection of positive control detecting agents. Positive control agents include binding agents or proteins/peptides that are known to bind a component of the sample, either a naturally occurring substance in the sample or a substance that is introduced into the sample to facilitate the positive control detection. In addition, one or more active areas on the surface of the biosensor contain a collection of negative control detecting agents. Negative control detecting agents include binding agents or proteins/peptides that should not bind to any possible substance present in the sample. In addition, one or more active areas on the surface of the biosensor contain no biological detecting agents immobilized on the surface of the graphene. The presence of active areas containing positive control, negative control, and no detecting agents allows for accurate detection and relative quantitation of the presence of true target molecules (e.g., antibodies or antigens) in the test sample via differential signal detection between the active areas containing the control detecting agents and no detecting agent and the areas containing the biological detecting agents.

Another aspect of the present disclosure is directed to a method of characterizing a pathogen's antigen profile. This method involves collecting a pathogen containing sample and providing the biosensor system as disclosed herein. Suitable biosensors include those containing a collection of different binding molecules immobilized at the active areas of the sensor. The method further involves delivering an electrical signal to the biosensor via the circuit of the electronic reader and determining a base resistance between the two or more electrodes at each active site on the biosensor. The method further involves applying the biological sample from the subject to the biosensor and identifying a change in the base resistance between the two or more electrodes at each active site on the biosensor resulting from said applying. The change in base resistance is indicative of a pathogenic protein from the sample binding to the immobilized binding molecule. The presence of the pathogen in the sample and/or the pathogen's antigenic profile can be characterized based on the identified change in base resistance between the electrode as the various active sites on the biosensor.

In some embodiments, the biosensor of the system comprises an electromagnet positioned beneath the substrate of the biosensor. In accordance with this embodiment, the methods of characterizing a subject's immune response or a pathogen's antigenic profile as described herein further comprise labeling target material present in the collected biological sample with a magnetic moiety. In some embodiments, the target material in the sample is antibodies present in the sample. In some embodiments, the target material in the sample is pathogen proteins and/or peptides. In some embodiments, the target material in the sample is a mixture of both antibodies (produced by the host subject) and pathogen proteins (derived from the infectious agent infecting or having infected the host subject). Regardless, the target material, either antibodies and/or proteins are labeled with a magnetic moiety. The biological sample containing the labeled antibodies and/or proteins is mixed in a viscous fluid to create a viscous biological sample mixture for applying to the biosensor. Once the sample is applied, the electromagnet is turned on to localize the labeled antibodies and/or proteins of the biological sample mixture to the active areas on the surface of the substrate to facilitate binding between labeled antibodies and their cognate pathogenic proteins or peptides immobilized on the active surface, between labeled proteins and their cognate binding molecules immobilized on the active surface, or between both. After allowing sufficient time for binding between the immobilized detection agents and magnetically labeled target material, the electromagnet is turned off to release unbound labeled antibodies and/or protein prior to identifying a change in the base resistance between the two or more electrodes at each active site on the biosensor.

In some embodiments, labeling the target material (antibodies or proteins) in a sample involves contacting the biological sample with an azide containing magnetic moiety, and exposing the contacted sample with UV light to conjugate the magnetic moiety to antibodies within the biological sample. In some embodiments, the magnetic moiety is a magnetic bead. Suitable magnetic beads include, without limitation, ferrous oxide magnetic bead. Suitable magnetic beads have a diameter of 2 nm to 100 um. For example, suitable magnetic beads have a diameter of 2 nm, 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 μm, 5 μm, 10 μm, 20 μm, 30 μm, 40 μm, 50 μm, 60 μm, 70 μm, 80 μm, 90 μm, or 100 μm.

The viscous fluid of containing the magnetically labeled target material that is applied to the biosensor surface can comprise any viscous fluid. Suitable viscous fluids include, without limitation fluids comprising polyethylene glycol (PEG) or glycerin. In some embodiments the viscous fluid comprises about 20% PEG, about 25% PEG, about 30% PEG, about 35% PEG, about 40% PEG, about 45% PEG, about 50% PEG, about 55% PEG, about 60% PEG, about 65% PEG, about 70% PEG, about 75% PEG, about 80% PEG, about 85% PEG, or about 90% PEG. Any PEG known in the art is suitable for use in accordance with this aspect of the disclosure. In some embodiments, the PEG is PEG-400.

### EXAMPLES

The examples below are intended to exemplify the practice of embodiments of the disclosure but are by no means intended to limit the scope thereof.

### Example 1— Comparison of Biologic Adhesion and Target Incubation on Graphene Silicon Sensor and Graphene Polyethylene Terephthalate (PET) Sensor Chips

To use these graphene surfaces as a diagnostic platform, the antibody or protein of interest is first adhered. This process (referred to herein as “adhesion”) starts with immobilizing a pyrene molecule to the surface of the graphene. Pyrene and graphene form a strong interaction through π-π stacking of the sp² carbons in each respective ring structure. Then each circuit undergoes a seven step adhesion process as described in Goldsmith et al., “Digital Biosensing by Foundry-Fabricated Graphene Sensors,” Sci. Rep. 9:434 (2019), which is hereby incorporated by reference in its entirety. This process involves activating the pyrene and then covalently attaching the protein or antibody of interest to the pyrene. The surface is then blocked and the reaction quenched by PEG-amine and ethanolamine, respectively. Following adhesion, the chips undergo washing prior to target incubation. In this portion of the experiment, the chips are calibrated by the addition of PBS buffer (a critical step for data normalization) followed by the addition of the antibody or protein of interest.

### Graphene Silicon Chips (Comparator)

Graphenea Silicon Chips (Graphena Chips) are individual graphene circuits purchased from Graphenea (Cambridge, Mass.). Each chip contains 12 circuits which can be used for multiplexing.

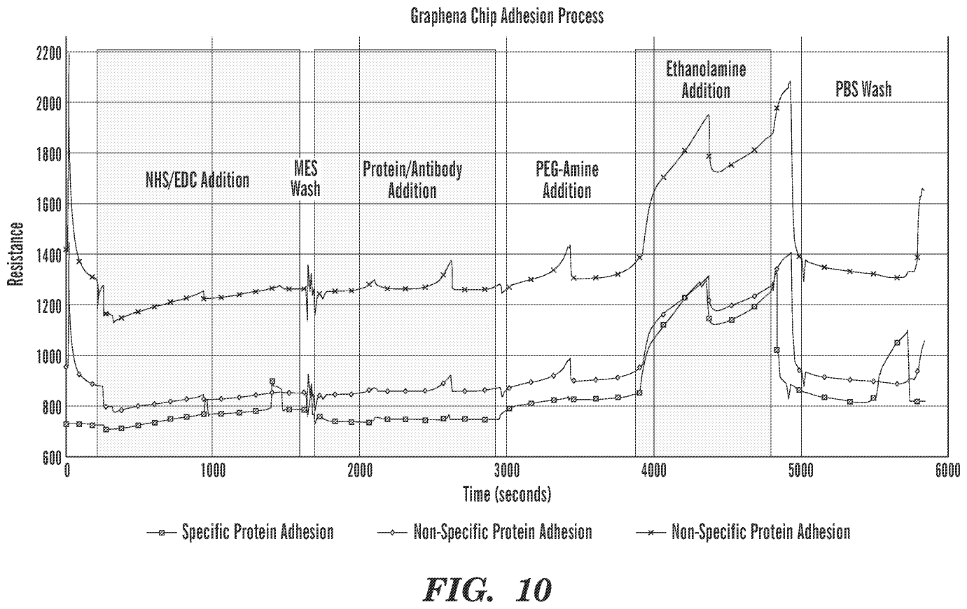

Adhesion: The adhesion process for BSA or Spike 1 protein on a Graphena chip followed the seven step process described above. A graph of circuit resistance over the 7-step adhesion process is shown in FIG. 10. Small jumps in the data occur when buffer is added to maintain surface saturation and are considered negligible when observing the entire protein adhesion process. Unfortunately, this method does not allow one to quantify the extent of protein or antibody surface adhesion. However, multiplexing is possible on these chips, which allows for the adhesion of multiple positive and negative controls in a single run. Additionally, Graphenea chips consistently show low standard deviations among circuits of the same run, providing confidence in the overall reliability of these circuits.

Target Incubation on the Graphenea Chips. Graphenea chips typically have low overall noise and a particularly good signal to noise ratio during the target incubation experiments. Specific Binding to the adhered BSA was tested with the addition of a BSA Antibody (1 mg/mL) to the circuit. Nonspecific Binding to the adhered Spike 1 protein was tested with the addition of BSA Antibody (1 mg/mL) to the circuit. The Blank Control is an unlabeled circuit with BSA Antibody (1 mg/mL) added to the circuit. The results, as summarized in Table 1 below, show a specific binding, non-specific binding, and blank control experiment conducted on one chip through multiplexing. This data is normalized using the PBS addition at −300 seconds in order to remove the effects of drift and accurately compare data. From this trial, selective binding is observed.

TABLE 1
Target Incubation on a Graphena Chip.

| Binding type | Normalized Resistance |
|---|---|
| Specific Binding | 400 ± 20 Ohms |
| Non-Specific Binding | 240 ± 30 Ohms |
| Blank Control | 150 ± 30 Ohms |

### General Graphene PET-Graphene Sheets

General Graphene PET-Graphene Sheets (PET-Graphene) were purchased as 4.5″×3.8″ sheets of PET plastic coated with a single layer of graphene. These sheets were cut into 0.7″×0.3″ rectangles and spotted with silver paint to establish electrical connection across the circuit (FIG. 11).

Adhesion: Experiments using a simplified 3 step adhesion process (eliminating blocking and quenching steps) confirmed the presence of adhered antibodies (BSA or pseudomonas) or protein using AFM and SEM on a silicon wafer. Using this three-step process on the PET-Graphene, the presence of protein was confirmed using a Coomassie Blue stain. Results from this adhesion process yields bound protein that was used for the following target incubation experiments.

Target Incubation on the PET-Graphene Chips: Target incubation in PET-Graphene initially showed promising results (see results in Table 2 below). There was a dose-response in the specific binding samples. Specific Binding (2×) to the adhered BSA antibody was tested with the addition of BSA protein (14 μg/mL) to the circuit. Specific Binding (1×) to the adhered BSA antibody was tested with the addition of BSA protein (7 μg/mL) to the circuit. Nonspecific Binding to the adhered Pseudomonas antibody with the addition of BSA protein (7 μg/mL) to the circuit. The Blank Control had nothing adhered (i.e., an unlabeled circuit) with BSA antibody (1 mg/mL) added to the circuit.

TABLE 2
Target Incubation with PET-Graphene Chips.

| Binding type | Normalized Resistance |
|---|---|
| Specific Binding (2X) | 3.5 ± 0.2 Ohms |
| Specific Binding (1X) | 2.9 ± 0.2 Ohms |
| Non-Specific Binding | 2.1 ± 0.3 Ohms |
| Blank | 1.3 ± 0.3 Ohms |

### Example 2: Electromagnetic Substrate Enhanced Target

Overview: As described herein the biosensors of the disclosure can contain an electromagnetic that is position beneath the biosensor surface. This electromagnet comprises an on/off switch to allow users to control the diffusion of the sample components, which have been magnetically labeled to the surface of the biosensor. The method of use generally involves using UV or chemical activation as known in the art to conjugate antibodies and/or proteins in a biological sample to magnetic moieties, such as magnetic beads. The labeled sample is mixed with a high density fluid to suspend magnetic bead-protein complex in solution and the mixture is added to the biosensor. FIG. 4A is a schematic showing the magnetically labeled target components of the sample applied to the biosensor when the electromagnet, positioned beneath the surface of the sensor is turned off.

When the electromagnet is turned on the magnetically labeled proteins and antibodies in the sample are brought in close proximity to the surface and active areas containing the detecting agents thereof as shown in FIG. 11B. This allows for target material in the sample to bind to its cognate binding partner (either an antibody, antibody-based molecule, or protein/peptide immobilized on the surface of the sensor).

Once time allowed for any binding interactions to occur has passed, the electromagnet is turned off to release the unbound magnetically labeled target material back into solution as shown in FIG. 11C below. The target material that is specifically bound to its immobilized binding partner on the surface remains bound to the surface, and a change in electrical current between circuits resulting from the binding of the target material specifically to its immobilized binding partner on the surface is measured.

Experimental Analysis: Blank circuits (die no. 56 on GG2) were rehydrated with 2.5 μL 80% PEG-400, 0.01×PBS. PEG-400 provides viscosity to the solution, which limits water evaporation (and therefore signal drift) and allows to probe binding kinetics (by slowing particle diffusion). Readings were obtained from two circuits. Two sets of ground magnets were placed under the wafer with the analyzed die centered on the magnet.

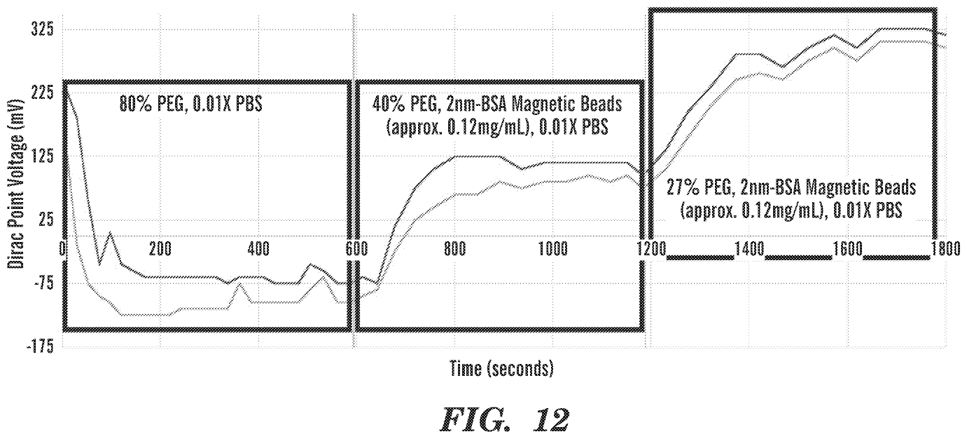

After initiation of the experiment, a steep decrease in signal is observed from 0-200 seconds which is likely due to signal stabilization (FIG. 12, far left box). Baseline signal is recorded from 200-600 seconds, with an average slope of 0.038. At 594 seconds, 2.5 μL of 2 nm-ferrous oxide magnetic beads conjugated to BSA in 0.01×PBS were added for a final concentration of 40% PEG-400, approx. 0.12 mg/mL BSA, 0.01×PBS. This addition led to an increase in signal from 600-800 seconds, with a slope of 1.09 (see FIG. 12, middle box). This transition stabilized at 800 seconds and baselined with a ΔV of approximately 200 mV until 1200 seconds (slope=0.0057). At 1188 seconds a second addition of 2.5 μL of 2 nm-ferrous oxide magnetic beads conjugated to BSA in 0.01×PBS was made for a final concentration of 27% PEG-400, approx. 0.12 mg/mL BSA, 0.01×PBS. This addition led to an increase in voltage by about 200 mV over the course of 200 seconds (slope=0.948) as shown in FIG. 12 (see far right box). The sample then baselined for the remaining 400 seconds (slope=0.154).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed is:

1. A biosensor comprising:
a substrate comprising a planar surface;
a spatially defined array of active areas on the planar surface of the substrate, each active area comprising:
a carbon material; and
at least two spaced electrodes, wherein the carbon material is deposited on the planar surface of the substrate between the at least two electrodes;
a plurality of pathogen proteins or peptides thereof, wherein different pathogen proteins or peptides thereof are positioned at different active areas and immobilized on the deposited carbon material of said active areas; and
an electrical connection comprising a plurality of electrical contacts, each electrical contact configured to transmit an electrical signal between the at least two electrodes of a single active area and the electrical connection.

2. The biosensor of claim 1, wherein the spatially defined array of active areas comprises at least 30 active areas.

3. The biosensor of claim 1 or claim 2, wherein the carbon material is graphene, carbon nanotube, or a combination thereof.

4. The biosensor of any one of claims 1-3, wherein the at least two electrodes comprise a conductive metal selected from Au, Cu and Ag.

5. The biosensor of any one of claims 1-4, wherein each active area further comprises a preservative solution.

6. The biosensor of any one of claims 1-5, wherein each of the plurality of pathogen proteins or peptides thereof is immobilized on the deposited carbon material via a hydrophobic linker, wherein said hydrophobic linker is coupled to a pathogen protein or peptide thereof via the protein or peptide's amino or carboxy terminus.

7. The biosensor of claim 6, wherein the hydrophobic linker is a peptide linker comprising two or more linker amino acids and one or more aromatic amino acid residues.

8. The biosensor of claim 7, wherein the two or more linker amino acid residues are selected from glycine, alanine, serine, and combinations thereof.

9. The biosensor of claim 6, wherein the hydrophobic linker comprises a polycyclic aromatic hydrocarbon.

10. The biosensor of any one of claims 1-9, wherein said biosensor further comprises:
a collection of antibody mimetics, wherein different antibody mimetics of the collection bind different pathogen proteins and wherein different antibody mimetics are positioned at different active areas not occupied by the pathogen protein or peptides, and wherein said antibody mimetics are immobilized on the deposited carbon material of said active areas.

11. The biosensor of any one of claim 1-10, wherein the pathogen is one or more infectious agents selected from a virus, a bacterium, or a combination thereof.

12. The biosensor of claim 11, wherein the pathogen is one or more viruses selected from SARS-CoV-2, Influenza A, Influenza B, Human papilloma virus, Venezuelan equine encephalitis virus, Vaccinia virus, Ebola virus, Lassa fever virus, Rift Valley fever virus and combinations thereof.

13. The biosensor of claim 12, wherein the one or more viruses is SARS-CoV-2.

14. The biosensor of claim 13, where the one or more viruses is SARS-CoV-2 and Influenza A.

15. The biosensor of claim 11, wherein the pathogen is one or more bacteria selected from Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.

16. The biosensor of any one of claims 1-13, wherein each of the plurality of pathogen peptides is between 5 and 50 amino acid residues in length.

17. The biosensor of any one of claims 1-16 further comprising:
an electromagnet positioned beneath the substrate of the biosensor.

18. The biosensor of any one of claims 1-17, wherein the substrate comprises a single-layer of anti-static polymeric material.

19. A biosensor system for characterizing a subject's immune response to pathogen exposure, the system comprising:
an electronic reader comprising:
a circuit for delivering a signal; and
a processing device for reading the signal;
a biosensor of any one of claims 1-18 operatively connected to the electronic reader via the electrical connection of the biosensor and configured to receive the signal delivered by the circuit; wherein the electronic reader is configured to deliver the signal to the biosensor and obtain an output impedance value before and after a sample has been applied to the array of active areas on the biosensor, and said processing device is configured to compare the output impedance values to determine whether a binding event has occurred at one or more of the active areas to characterize the subject's immune response to pathogen exposure.

20. The biosensor system of claim 19, where in the biosensor comprises an electromagnet positioned beneath the substrate of the biosensor.

21. The biosensor system of claim 19 further comprising:
a communication interface coupled to the electronic reader for transmitting data from the electronic reader;
a data management computing device configured to receive data from the electronic reader via the communication interface, said data management computing device comprising a memory coupled to a processor which is configured to execute programmed instructions comprising and stored in the memory to:
geographically map immune response data to pathogen exposure, based on data received from electronic reader.

22. A method of characterizing a subject's immune response to pathogen exposure, said method comprising:
collecting a biological sample from a subject;
providing the biosensor system of any one of claims 19-21;
delivering an electrical signal to the biosensor via the circuit of the electronic reader;
determining a base resistance between the two or more electrodes at each active site on the biosensor;
applying the biological sample from the subject to the biosensor;
identifying a change in the base resistance between the two or more electrodes at each active site on the biosensor resulting from said applying;
characterizing the subject's immune response to said pathogen exposure based on said identifying.

23. The method of claim 21, wherein the biosensor of the system comprises an electromagnet positioned beneath the substrate of the biosensor, said method further comprising:
labeling, after said collecting, antibodies present in the biological sample with a magnetic moiety;
mixing the biological sample containing the labeled antibodies with a viscous fluid to create a viscous biological sample mixture for said applying;
turning on said electromagnet to localize the labeled antibodies of the biological sample mixture to the active areas on the surface of the substrate during said applying to facilitate binding between labeled antibodies and their cognate pathogenic proteins or peptides immobilized on the active surface; and
turning off said electromagnet to release unbound labeled antibodies prior to said identifying.

24. The method of claim 23, wherein said labeling comprises:
contacting the biological sample with an azide containing magnetic moiety, and
exposing the contacted sample with UV light to conjugate the magnetic moiety to antibodies within the biological sample.

25. The method of claim 24, wherein the magnetic moiety is a magnetic bead.

26. The method of claim 25, wherein the magnetic bead is a ferrous oxide magnetic bead.

27. The method of claim 25, wherein the magnetic bead has a diameter of 2 nm to 100 um.

28. The method of claim 23, wherein the viscous fluid comprises polyethylene glycol (PEG) or glycerin.

29. The method of claim 28, wherein the PEG is PEG-400.

30. The method of claim 28, wherein the viscous fluid comprises about 20% to about 90% PEG.

31. A biosensor comprising:
a substrate comprising a planar surface;
a spatially defined array of active areas on the planar surface of the substrate, each active area comprising:
a carbon material; and
at least two spaced electrodes, wherein the carbon material is deposited on the planar surface of the substrate between the at least two electrodes;
a collection of binding molecules, wherein different binding molecules of the collection bind different pathogen proteins and wherein different binding molecules are positioned at different active areas and immobilized on the deposited carbon material of said active areas; and
an electrical connection comprising a plurality of electrical contacts, each electrical contact configured to transmit an electrical signal between the two or more electrodes of a single active area and the electrical connection.

32. The biosensor of claim 31, wherein the spatially defined array of active areas comprises at least 30 active areas.

33. The biosensor of claim 31 or claim 32, wherein the carbon material is graphene, carbon nanotubes, or a combination thereof.

34. The biosensor of any one of claims 31-33, wherein the at least two electrodes comprise a conductive metal selected from Au, Cu, and Ag.

35. The biosensor of any one of claims 31-34, wherein each active area further comprises a preservative solution.

36. The biosensor of any one of claims 31-35, wherein each binding molecule of the collection is immobilized on the deposited carbon material via a hydrophobic linker, wherein said hydrophobic linker is coupled to a binding molecule via the binding molecule's amino or carboxy terminus.

37. The biosensor of claim 36, wherein the hydrophobic linker is a peptide linker comprising two or more linker amino acid residues and one or more aromatic amino acid residues

38. The biosensor of claim 37, wherein the two or more linker amino acid residues are selected from glycine, alanine, serine, and combinations thereof.

39. The biosensor of claim 36, wherein the hydrophobic linker comprises a polycyclic aromatic hydrocarbon.

40. The biosensor of any one of claim 31-39, wherein the pathogen is one or more infectious agents selected from a virus, a bacterium, a toxin, or combinations thereof.

41. The biosensor of claim 40, wherein the pathogen is one or more viruses selected from SARS-CoV-2, influenza A, influenza B, human papilloma virus, Venezuelan equine encephalitis virus, Vaccinia virus, Ebola virus, Lassa fever virus, Rift Valley fever virus and combinations thereof.

42. The biosensor of claim 41, wherein the one or more viruses is SARS-CoV-2.

43. The biosensor of claim 41, wherein the one or more viruses is SARS-CoV-2 and Influenza A.

44. The biosensor of claim 40, wherein the pathogen is one or more bacteria selected from Pseudomonas aeruginosa, Neisseria gonorrhoeae, Chlamydia trachomatis, Treponema pallidum, Bacillus anthracis, Yersinia pestis, Francisella tularensis, Burkholderia pseudomallei, Burkholderia mallei, and combinations thereof.

45. The biosensor of claim 40, wherein the pathogen is one or more toxins selected from Ricin toxin, Botulinum Toxin A/B/E, Staphylococcus enterotoxin B (SEB), Abrin toxin, T-2 toxin, B. anthracis LF toxin, B. anthracis EF toxin, B. anthracis PA toxin, and combinations thereof.

46. The biosensor of any one of claims 31-45, wherein the binding molecules of the collection are antibody mimetics.

47. The biosensor of any one of claims 31-45, wherein the antibody mimetics are selected from the group consisting of an affibodies, affilins, affimers, monobodies, and DARPINs.

48. The biosensor of any one of claims 31-45, wherein the binding molecules of the collection are antibody-based molecules.

49. The biosensor of claim 48, wherein the antibody-based molecules are selected from antibodies, epitope-binding domains thereof, and antibody derivatives.

50. The biosensor of any one of claims 31-49 further comprising:
an electromagnet positioned beneath the substrate of the biosensor.

51. The biosensor of any one of claims 31-49, wherein the substrate comprises a single-layer of anti-static polymeric material.

52. A biosensor system for characterizing a pathogen's antigen profile, the system comprising:
an electronic reader comprising:
a circuit for delivering a signal and
a processing device for reading the signal;
a biosensor of any one of claims 31-50 operatively connected to the electronic reader via the electrical connection of the biosensor and configured to receive the signal delivered by the circuit; wherein the electronic reader is configured to deliver the signal to the biosensor and obtain an output impedance value before and after a sample has been applied to the array of active areas of the biosensor, and said processing device is configured to compare the output impedance values to determine whether a binding event has occurred at one or more of the active areas to characterize the pathogen's antigen profile.

53. The biosensor system of claim 52, where in the biosensor comprises an electromagnet positioned beneath the substrate of the biosensor.

54. The biosensor system of claim 52 or claim 53 further comprising:
a communication interface coupled to the electronic reader for transmitting data from the electronic reader;
a data management computing device configured to receive data from the electronic reader via the communication interface, said data management computing device comprising a memory coupled to a processor which is configured to execute programmed instructions comprising and stored in the memory to:
geographically map pathogen antigen, based on data received from electronic reader.

55. A method of characterizing a pathogen's antigen profile, said method comprising:
collecting a pathogen containing sample;
providing the biosensor system of any one of claims 52-54;
delivering an electrical signal to the biosensor via the circuit of the electronic reader;
determining a base resistance between the two or more electrodes at each active site on the biosensor;
applying the pathogen containing sample to the biosensor;
identifying a change in the base resistance between the two or more electrodes at each active site on the biosensor resulting from said applying;
characterizing the pathogen's antigen profile based on said identifying.

56. The method of claim 55, wherein the biosensor of the system comprises an electromagnet positioned beneath the substrate of the biosensor, said method further comprising:
labeling, after said collecting, proteins present in the biological sample with a magnetic moiety;
mixing the biological sample containing the labeled proteins with a viscous fluid to create a viscous biological sample mixture for said applying;
turning on said electromagnet to localize the labeled proteins of the biological sample mixture to the active areas on the surface of the substrate during said applying to facilitate binding between labeled proteins and their cognate binding molecule immobilized on the active surface; and
turning off said electromagnet to release unbound labeled proteins prior to said identifying.

57. The method of claim 56, wherein said labeling comprises:
contacting the biological sample with an azide containing magnetic moiety, and
exposing the contacted sample with UV light to conjugate the magnetic moiety to proteins within the biological sample.

58. The method of claim 56, wherein the magnetic moiety is a magnetic bead.

59. The method of claim 57, wherein the magnetic bead is a ferrous oxide magnetic bead.

60. The method of claim 58, wherein the magnetic bead has a diameter of 2 nm to 100 um.

61. The method of claim 56, wherein the viscous fluid comprises polyethylene glycol (PEG) or glycerin.

62. The method of claim 61, wherein the PEG is PEG-400.

63. The method of claim 61, wherein the viscous fluid comprises about 20% to about 90% PEG.
